(19) United States
(12) Patent Application Publication
(10) Pub. No.: US 2012/0330361 A1
(43) Pub. Date: Dec. 27, 2012

(54) SPINAL IMPLANTABLE DEVICES MADE OF CARBON COMPOSITE MATERIALS AND USE THEREOF

(76) Inventor: Reuven Gepstein, Kfar Saba (IL)

(21) Appl. No.: 13/582,756

(22) PCT Filed: Mar. 10, 2011
(86) PCT No.: PCT/IL11/00233

§ 371 Date: Sep. 5, 2012

Related U.S. Application Data
(60) Provisional application No. 61/312,565, filed on Mar. 10, 2010.

(51) Int. Cl. A61B 17/86; A61B 17/80; A61B 17/68; A61B 17/56; A61B 17/70

(52) U.S. Cl. 606/254; 606/76; 606/301; 606/246; 606/256; 606/60; 606/298

(57) ABSTRACT

A spinal implantable device may include composite material comprising matrix including PEEK. The PEEK matrix may be reinforced with carbon fibers that amount to at least 60% of the composite material. The carbon fibers are arranged in a substantially parallel arrangement and compressed in a direction perpendicular to a longitudinal direction of the carbon fibers.

### BACKGROUND OF THE INVENTION

Spinal fusion is a common surgery for treatment of spinal pathologies. Typically, metal implants are used for this purpose—intra-pedicular screws, hooks and rods. However, even after major surgery, about 20%-30% of patients continue to suffer. In such cases, the patient may feel worse than before because no further options are available.

The cause of this failure is not known. Current imaging techniques are not sufficient to reveal the cause of such failure. Computed tomography (CT) imaging may not give good visualization of the areas of interest due to masking of the metal implants located near the pathology (nerves, discs, joints, etc.). Using Magnetic Resonance Imaging (MRI) may be inappropriate because of the existence of metal implants in the patient's body near the pathology, masking the anatomy. Moreover, follow up of the surgery for evaluation of tumor expansion, deterioration in oncology cases, or evaluation of bone fusion is also blocked by the metallic artifacts in all imaging techniques. All of this may lead a spinal surgeon to perform second and third operations in order to remove the metal implants, obtain a better image of the pathology so as to determine causes of the failure and decide on appropriate treatment.

A possible solution to this considerable problem is to use implants made of a composite material instead of metallic implants. Composite material implants, such as Carbon fibers reinforced PolyEtherEtherKetone (PEEK) implants do not interfere with imaging techniques and allow clear view which is required for evaluation of post operation conditions. Moreover, composite materials have better elasticity than metal implants, and can adapt to the patient's individual condition and pathology. Due to the similarity of the elasticity of composite materials to the elasticity of bone, stress shielding phenomena is less likely to occur, which may lead to fewer stress fractures of implants and bone and fewer loosening of screws. Hence, in some cases, a bone graft may not be necessary in dynamic rod usage, such as in spinal fixation mode.

Although composite carbon polymer materials are very strong (for example, carbon reinforced PEEK may be five times stronger than metal), and are commonly used in the aircraft industry, these materials have also been used in spine surgery (e.g. carbon PEEK cages). However, intra-pedicular screws, hooks and reinforced rods for spinal fusion have not been made of composite materials so far.

For example, the following products are available for use in treatment of the spine: Spine system with composite rods made of Carbon-PEEK, and metal screws manufactured by coLigne International. Spine system with PEEK rods manufactured by Expedium spine system, DePuy. Spine system with rods made of metal cable coated with PEEK, manufactured by Biomech. Carbon PEEK cage: Aesculap-ProSpace PEEK.

Spinal stenosis, or narrowing of the spinal canal—soft tissue and bony stenosis—is a very common spinal disorder of the elderly. Surgical treatment for this condition is commonly applied, typically including open surgery decompression of the stenotic spinal canal.

### SUMMARY OF THE INVENTION

According to embodiments of the present invention there is provided a spinal implantable device. The device may include composite material comprising matrix including PEEK, reinforced with carbon fibers that amount to at least 60% of the composite material, wherein said carbon fibers are arranged in a substantially parallel arrangement and compressed in a direction perpendicular to a longitudinal direction of the carbon fibers.

Furthermore, according to embodiments of the present invention, the spinal implantable device may be a screw comprising a central shaft made of the composite material, wherein the carbon fibers stretch along a longitudinal axis of the central shaft.

Furthermore, according to embodiments of the present invention, the screw may further include threads and screw tip made of said composite material.

Furthermore, according to embodiments of the present invention, the screw may further include a coating made of a rigid material wherein the coating may include threads and tip of said screw.

Furthermore, according to embodiments of the present invention, the coating may be made by laser welding of an outer coating layer made of the rigid material around the central shaft.

Furthermore, according to embodiments of the present invention, the coating may be made by producing a secondary screw of the rigid material, removing an area corresponding to the central shaft from the center of the secondary screw, leaving an outer shell made of the rigid material, wherein the outer shell may include threads and tip of said screw and filling the outer shell with the composite material.

Furthermore, according to embodiments of the present invention, the rigid material may be selectable from a list including: titanium, Hydroxyapatite and metal.

Furthermore, according to embodiments of the present invention, the screw may further include a hole through a center of the screw, along the longitudinal axis of the screw.

Furthermore, according to embodiments of the present invention, the screw may be capable of flexing to an angle of 6 degrees.

Furthermore, according to embodiments of the present invention, the spinal implantable device may be a rod made of the composite material, wherein the carbon fibers stretch along a longitudinal axis of the rod.

Furthermore, according to embodiments of the present invention, the rod may further be capable of flexing to an angle of 6 degrees.

Furthermore, according to embodiments of the present invention, the rod may further include a joint.

Furthermore, according to embodiments of the present invention, the spinal implantable device may further be a cup made of the composite material, wherein the carbon fibers stretch along a circumference of the cup.

Furthermore, according to embodiments of the present invention, the spinal implantable device may further be a plate made of the composite material, wherein the carbon fibers stretch along a longitudinal axis of the plate.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1A depicts an exemplary screw according to embodiments of the present invention;

FIG. 1B shows a cross-section along the length of the exemplary screw shown in FIG. 1A, and compression direction of carbon fibers according to embodiments of the present invention;

FIG. 1C shows a cross section across the width of the exemplary screw shown in FIG. 1A, and compression direction of carbon fibers according to embodiments of the present invention;

FIG. 2A depicts an exemplary plate according to embodiments of the present invention;

FIG. 2B shows a cross section across the depth of the exemplary plate shown in FIG. 2A and compression direction of carbon fibers according to embodiments of the present invention;

FIG. 2C shows a cross section across the length of the exemplary plate shown in FIG. 2A and compression direction of carbon fibers according to embodiments of the present invention;

FIG. 2D shows a cross section across the width of the exemplary plate shown in FIG. 2A and compression direction of carbon fibers according to embodiments of the present invention;

FIG. 3A depicts an exemplary cup according to embodiments of the present invention;

FIG. 3B shows a cross-section along the length of the exemplary cup shown in FIG. 3A, and compression direction of carbon fibers according to embodiments of the present invention;

FIG. 3C shows a cross-section along the width of the exemplary cup shown in FIG. 3A, and compression direction of carbon fibers according to embodiments of the present invention;

FIG. 4A depicts an exemplary rod with flexibility along its longitudinal direction according to embodiments of the present invention;

FIG. 4B depicts the exemplary rod shown in FIG. 4A in bended position according to embodiments of the present invention;

FIG. 4C depicts an enlarged cross-sectional view of the exemplary rod shown in FIG. 4A according to embodiments of the present invention;

FIG. 4D depicts an exemplary rod with a joint according to embodiments of the present invention;

FIG. 5 depicts a cross-sectional view of a main body of an exemplary screw coated with rigid material according to embodiments of the present invention;

FIG. 6A depicts method for coating screw with rigid coating according to embodiments of the present invention;

FIG. 6B depicts another method for coating screw with rigid coating according to embodiments of the present invention;

FIG. 7 depicts a screw according to embodiments of the invention, adapted to be used in minimally invasive surgery; and

FIG. 8 is a flowchart illustration of a--method for making a composite material screw according to embodiments of the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Although embodiments of the present invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. Unless explicitly stated, the method embodiments described herein are not constrained to a particular order or sequence. Additionally, some of the described method embodiments or elements thereof can occur or be performed at the same point in time.

In accordance with embodiments of the present invention, implantable devices for the spine, for procedures such as spinal fusion surgeries, including (but not limited to) screws such as intra-pedicular screws, hooks, cups, plates, rods and locking devices for rods may be made of composite materials such as carbon polymer composite materials. Such carbon polymer composite materials may include PEEK reinforced typically with at least 60% carbon fibers. For example, such composite materials may include 60%-80% carbon fibers embedded in 20%-40% PEEK. High percentage of carbon fibers in a composite material may provide a composite material having low weight but high tensile and compressive strength and stiffness along the longitudinal (fiber) direction. The orientation of the fibers may be controlled to ensure maximal tensile and compressive strength in desired directions.

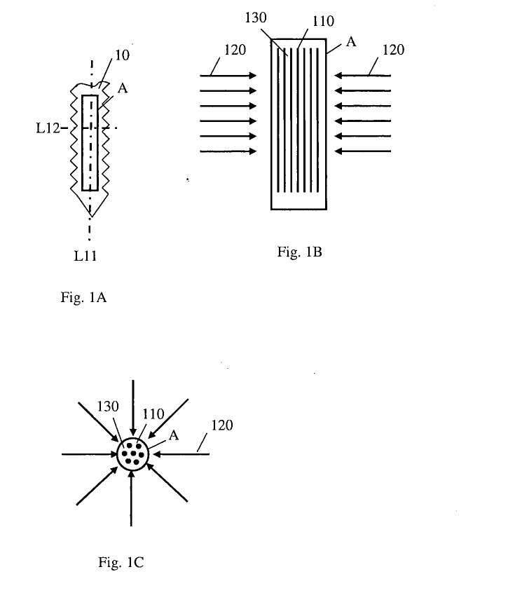

Reference is made to FIG. 1A depicting an exemplary screw 10 and to FIGS. 1B-C depicting cross-sectional views of screw 10 and compression direction of carbon fibers 110 according to embodiments of the present invention. FIG. 1A depicts an exemplary screw 10, such as, but not limited to an intra-pedicular screw. FIG. 1B depicts a cross-sectional view of void A within screw 10, including the composite material part of screw 10, along axis L11. FIG. 1C depicts a cross-sectional view of void A within screw 10 made along axis L12, at right angle to axis L11. According to embodiments of the present invention, screw 10 may include PEEK 130 reinforced with at-least 60% carbon fibers 110. According to embodiments of the present invention, carbon fibers 110 may be placed in a substantially parallel arrangement (parallel to each other) stretching along the longitudinal axis of screw 10. During the curing phase of the manufacturing process, pressure may be applied in a direction perpendicular to the orientation of carbon fibers 110 in the inward radial direction, such that carbon fibers 110 may be compressed in a direction perpendicular to a longitudinal direction of carbon fibers 110, as indicated by arrows 120. Optionally, carbon fibers 110 may be washed after being placed in PEEK 130 matrix and before being compressed. It should be noted that FIGS. 1B and 1C may represent carbon fiber orientation and pressure direction related to any substantially cylindrical implantable devices such as screw 10 as well as a rods, according to embodiments of the present invention.

According to embodiments of the present invention producing screws and rods from at least 60% carbon fibers reinforced PEEK; arranging the carbon fibers 110 in a longitudinal orientation arrangement, as depicted in FIGS. 1B-C and applying pressure in a direction perpendicular to the orientation of carbon fibers 110 in the inward radial direction, as indicated by arrows 120, during the manufacturing process, may result in the provision of implantable devices such as rods and screws that are characterized by high tensile and compressive strength along the longitudinal direction (L11 in FIG. 1A), enabling the screws and rods to sustain high bending forces in the direction of arrows 120 (see FIG. 1B), as may be required from such devices after implantation.

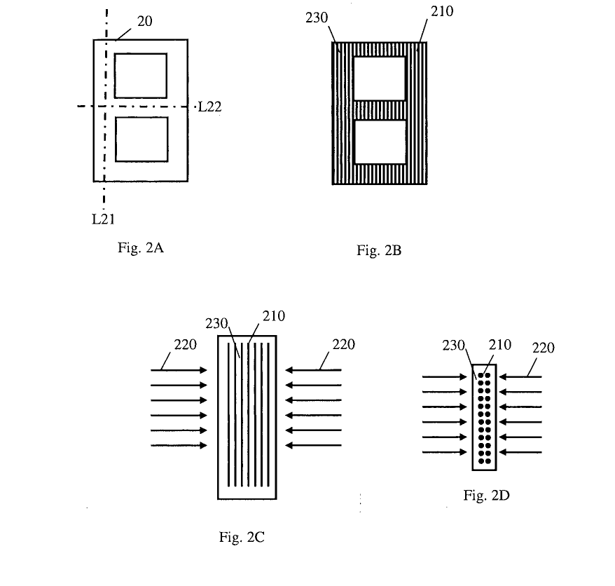

Reference is made to FIG. 2A depicting an exemplary plate 20 and to FIGS. 2B-D depicting cross-sectional views of exemplary plate 20 and compression direction of carbon fibers 210 according to embodiments of the present invention. FIG. 2A depicts an exemplary plate 20, FIG. 2B depicts a cross-sectional view of plate 20, across the depth of the plate 20, FIG. 2C depicts a cross-sectional view of plate 20 made along axis L21, and FIG. 2D depicts a cross-sectional view of plate 20 made along axis L22, at right angle to axis L21. According to embodiments of the invention plate 20 may include PEEK 230 reinforced with at-least 60% carbon fibers 210. According to embodiments of the present invention, carbon fibers 210 may be substantially straight, parallel to each other, and stretch along the longer side of plate 20. During the curing phase of the manufacturing process pressure may be applied in a direction perpendicular to the orientation carbon fibers 210, as indicated by arrows 220, carbon fibers 210 may be compressed in a direction perpendicular to a longitudinal direction of carbon fibers 210. Optionally, carbon fibers 210 may be washed after being placed in PEEK 230 matrix and before being compressed.

Similarly to the screws and rods, plate 20 may exhibit high tensile and compressive strength along the longitudinal direction of the fibers, marked as L21, enabling plate 20 to sustain high bending forces in the direction of arrows 220, as may be required form such devices after implantation.

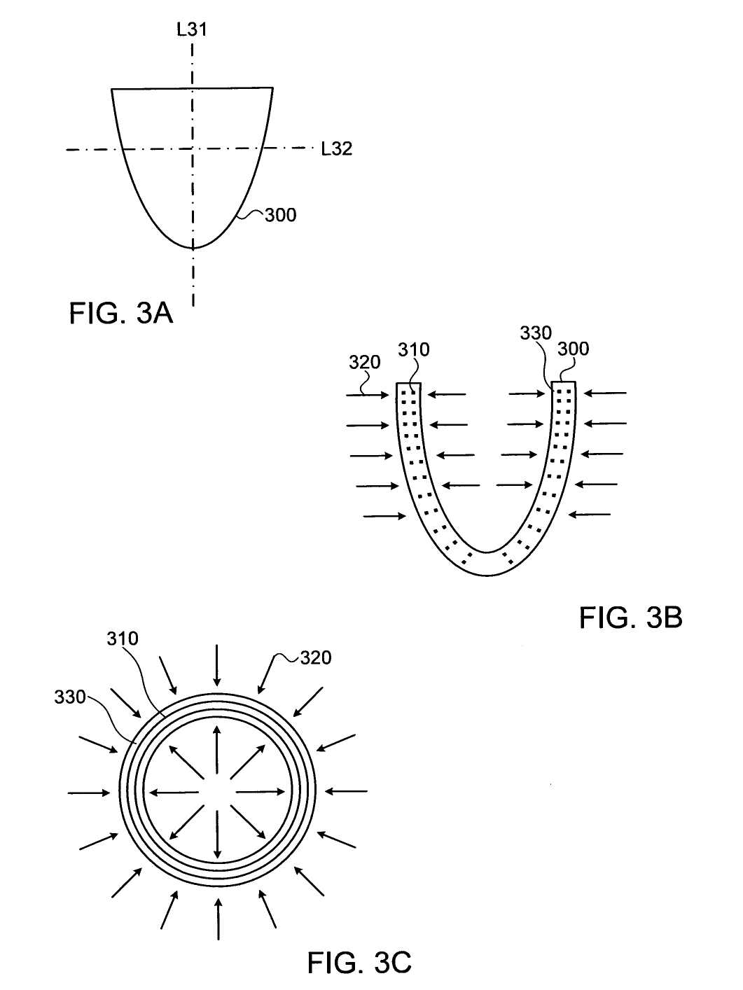

Reference is made to FIGS. 3A depicting an exemplary cup 300 and to FIGS. 3B-C depicting cross-sectional views of an exemplary cup 300 and compression direction of carbon fibers 310 according to embodiments of the present invention. FIG. 3A depicts an exemplary cup 300, FIG. 3B depicts a cross-sectional view of cup 300 along Axis L31, and FIG. 3C depicts a cross-sectional view of cup 300 made along axis L32, at right angle to axis L21. According to embodiments of the present invention cup 300 may include PEEK 330 reinforced with at-least 60% carbon fibers 310. According to embodiments of the present invention, carbon fibers 310 may be substantially concave, parallel to each other and stretch along the circumference of plate 300. During the curing phase of the manufacturing process pressure may be applied in a direction perpendicular to the orientation of carbon fibers 310, as indicated by arrows 320, such that carbon fibers 310 may be compressed in a direction perpendicular to the orientation of carbon fibers 310.

Optionally, carbon fibers 310 may be washed after being placed in PEEK 330 matrix and before being compressed.

Similarly to the screws and rods, cup 300 may exhibit high tensile and compressive strength along the longitudinal direction of the fibers, that is, along the circumference of cup 300, enabling cup 300 to sustain high bending forces in the direction of arrows 320, as may be required form such devices after implantation.

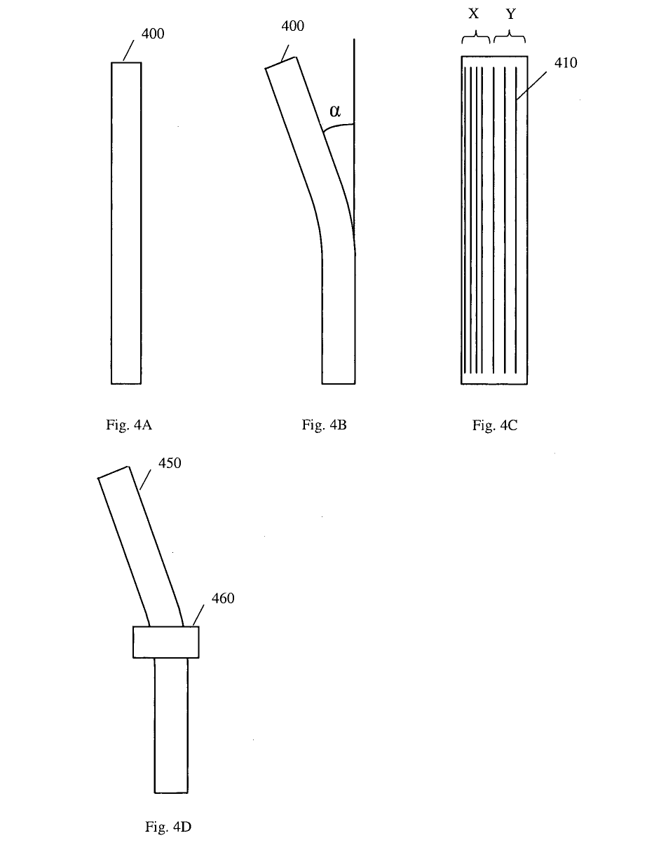

Reference is now made to FIGS. 4A-D depicting an exemplary rod 400 with flexibility along its longitudinal direction according to embodiments of the present invention. FIG. 4A depicts an exemplary rod 400, FIG. 4B depicts rod 400 in bended position and FIG. 4C depicts an enlarged cross-sectional view of rod 400 demonstrative organization of carbon fibers 410. Implantable devices made of at least 60% carbon fibers reinforced PEEK according to embodiments of the invention, may have a certain flexibility along their longitudinal direction. For example, rod 400 may flex to an angle a, for example up to 6 degrees or up to 10 degrees, as may be required for the medical application. The level of flexibility given to implantable devices such as rods and screw according to embodiments of the present invention may depend on the density and organization of carbon fibers 410. For example, higher density of carbon fibers 410 at side X of rod 400 and lower density of carbon fibers 410 at side Y of rod 400 may cause side Y to yield and stretch more under tensile forces and therefore under bending forces, rod 400 may bend in the direction of the dense fibers, as indicated in FIG. 4B. For example, side X of rod 400 may include carbon fibers that amount to more than 60% of the composite material and side Y of rod 400 may include carbon fibers that amount to less than 60% of the composite material. Additionally, rods or plates may be made with elasticity or motion, for example, a joint adapted to individual pathologies such as instability, tumors, trauma, scoliosis, degenerative conditions, etc.

Reference is now made to FIG. 4D depicting an exemplary rod 450 with a joint 460 according to embodiments of the present invention. rod 450 made of at least 60% carbon fibers reinforced PEEK according to embodiments of the invention may include a joint 460 to enable dynamization of a fixation system.

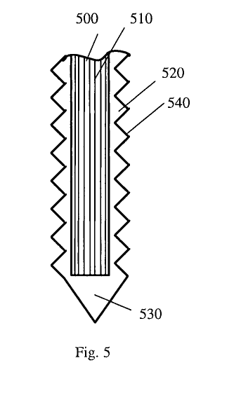

Reference is now made to FIG. 5 depicting a cross-sectional view of a main body of an exemplary screw 500 coated with rigid material 520 according to embodiments of the present invention. As known in the art threads of screws made of composite materials such as carbon reinforced PEEK may break while screwed to a bone such as a vertebra. This is due to a relative weakness of the threads of the composite material screws. According to embodiments of the present invention of screw 500 may include a shaft 510 made of at least 60% carbon fibers reinforced PEEK, coated with coating 520 made of rigid material such as Hydroxyapatite or titanium or metallic or non metallic rigid materials wherein coating 520 includes threads 540. Additionally, screw tip 530 may also be made of such rigid material for reinforcement. Such materials may not brake while screw 500 is screwed. Additionally Hydroxyapatite and titanium are considered biocompatible and when made very thin may substantially not interfere, or interfere very little, with CT and MRI imaging allowing post surgery follow-up. It should be noted that coating 520 may be made from any other material that is bio-compatible, rigid and allows imaging by high resolution imaging techniques such as CT and MRI. For example, screw 500 may be partially coated with metallic material when used at sites which are not near pathology or nerves, and in small quantities so as not to interfere with imaging techniques.

Alternatively, a taper may be used (not shown) to drill a hole in the vertebra for the screw, prior to screwing the screw. The screw may be screwed after removing the taper, applying relatively low force on the threads of the screw. If a taper is used for drilling a hole for the screw, the screw may be made from carbon fibers reinforced PEEK only.

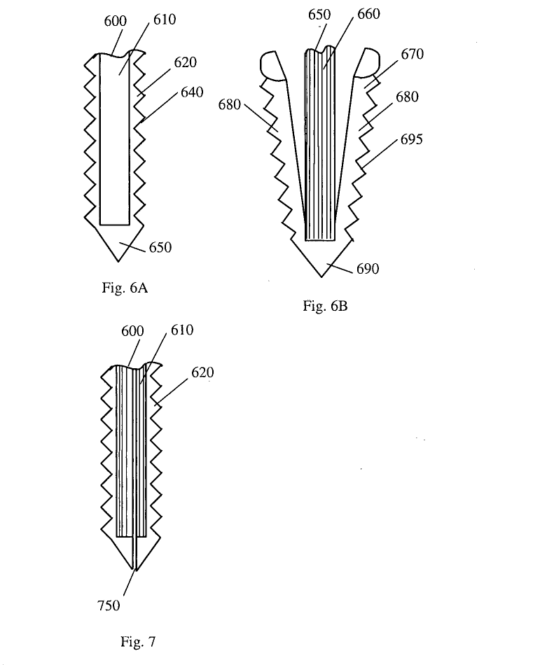

Reference is now made to FIGS. 6A-B depicting methods for coating screw with rigid coating according to embodiments of the present invention. FIG. 6A depicts a secondary screw 600 made of a rigid material such as titanium. According to embodiment of the preset invention, secondary screw 600 may be made entirely from titanium. An area corresponding to shaft area 610 of screw 600 may be removed using any suitable method, as known in the art, leaving an outer thin shell 620 wherein outer shell 620 may include threads 640 and screw tip 650 of screw 600. Outer shell 620 may be filled with at least 60% carbon fibers reinforced PEEK oriented and fabricated as described above. The final screw may have carbon reinforced PEEK shaft and rigid material coating, as shown in FIG. 5. FIG. 6B depicts a screw 650 made of a central shaft 660 made of at least 60% carbon fibers reinforced PEEK oriented and fabricated as described above, to which an outer coating layer 670, made of thin layer of rigid material may be composed. Coating layer 670 may include threads 695 and screw tip 690 of screw 650 and may include at least two sheets 680 that may be welded together around shaft 660 using any suitable method as known in the art, such as, for example, laser welding. All methods of production and methods of use mentioned above are suitable for spinal instrumentations including screws, rods, plates, cages and cables.

It should be noted that the screws, rods, plates, and cups are presented here by way of example only, and that other implantable devices used for lumbar, thoracic and cervical areas of the spine having various geometries as know in the art may be made according to embodiments of the preset invention as described herein. For example, FIG. 7 depicts a screw 700 according to embodiments of the invention, adapted to be used in minimally invasive surgery. Screw 700 may include a central shaft made of at least 60% carbon fibers reinforced PEEK oriented and fabricated as described above with a rigid coating 720. Screw 700 may be cannulated to suit minimally invasive surgery by drilling a hole 750 through the center of screw 700 along the longitudinal axis of screw 700 for a guidewire, such as Kirscher “k” wire, thus enabling percutaneous insertion of screw 700.

Implantable devices made according to embodiments of the present invention such as screws, hooks, plates, cables, cages and rods for lumbar, thoracic and cervical areas, including plates and screws for anterior or posterior approach of all sections of the spine: from two levels up to scoliosis treatment of a large spinal area (the whole spine). The screws can also include tunnels (holes) to enable bone integration within the screws, and roughening of the surface such as coated carbon to promote engagement of the screws or plate to the bone, as well as bone ingrowth.

Rods and screws made according to embodiments of the present invention may include radio-opaque materials to enable evaluation and follow up of the post-operative position and function with imaging techniques.

Diameters of implantable devices in accordance with embodiments of the present invention may be similar to those of existing metal implants or smaller due to the fact that composite material is stronger than titanium and hence the surgical technique will be easier and safer (less morbidity). All systems may enable percutaneous or open surgery, posterior or anterior approach. Rods may be supplied in bended forms as needed clinically to adjust the anatomical curves of the spine.

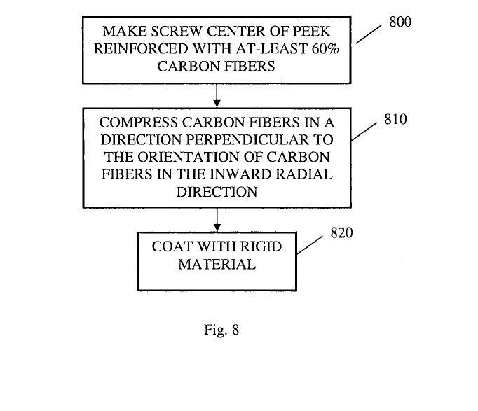

Reference is now made to FIG. 8 which is a flowchart of a method for making a composite material screw according to embodiments of the present invention. According to embodiments of the present invention, a central part of screw may be made of composite material including matrix including PEEK, the matrix reinforced with carbon fibers that amount to at least 60% of the composite material, as indicated in block 800. The carbon fibers may be substantially straight and parallel to each other and stretch along the longitudinal axis of the screw. The carbon fibers may be placed together with the PEEK matrix in a metal frame. Optionally the carbon fibers may be washed. During the curing phase of the manufacturing process, pressure may be applied in a direction perpendicular to the orientation of carbon fibers in the inward radial direction such that the carbon fibers may be compressed in a direction perpendicular to a longitudinal direction of the carbon fibers, as indicated in block 810. In block 820 the screw may be coated with a rigid material, forming a frame to the carbon fibers that may include the threads and screw tip of the screw. The rigid material may be selected, for example, to be titanium or Hydroxyapatite.

In a method of treatment, in accordance with embodiments of the present invention, decompression of soft and bony tissue around spinal dura within the spinal canal is performed in a percutaneous minimally invasive surgery, using a tool which is maneuverable so as to approach the inner spinal canal boundaries. Instruments that may be used for such a minimally invasive procedure may include, for example, an instrument designed for sinus surgery, possibly modified to adapt to varying spinal anatomy and sizes and to provide further protection to avoid neural tissue damage (the work is within the spinal canal).

Moreover, an irrigation and suction system will be operated for flushing and evacuating debris outside the spinal canal. The system may be a closed system and connected to the instruments since all the surgery is percutaneus.

The instruments used may be variations of instruments such as: Arthronet—arthronet Germany LTD &Co KG.D-51399 Burscheid. Medtronic powered surgical equipment and accessories—XPS Straight Sinus Blades

The instruments may optionally include 2 tubes (diameter 2-4 mm): one external which is static and includes a window, and one internal that rotates within the external tube and with an additional sharp-edged window. The inner tube is provided with opening and sharp edges that ablate the soft and bony tissue around the dura without the necessity of open surgery. Thus, the spinal canal may be decompressed and enlarged, leaving more space for the neural tissue.

The method of treatment may enable decompression of the spinal canal without necessitating open surgery. It can be preformed under local or general anesthesia, for example, through a 2 to 4 mm key hole in the skin, avoiding excessive bleeding, or damage to tissue, muscles, ligaments, bone or joints, that may be caused by open surgery.

All debris may be flushed out through a closed system, under vacuum irrigation.

Patients may be discharged immediately post operatively; no or little rehabilitation may be needed. Surgery may be performed with the assistance of an image intensifier and/or endoscopic equipment.

Thus, a new method of treatment is described in which decompression (wide) is performed through small hole (2-4 mm) under local or general anesthesia. It can be performed in all spinal areas (lumbar and cervical), avoiding open surgery with the complications associated with anesthesia and open surgery.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A spinal implantable device, comprising:
composite material comprising matrix including PEEK, said matrix reinforced with carbon fibers that amount to at least 60% of the composite material,
wherein said carbon fibers are arranged in a substantially parallel arrangement and compressed in a direction perpendicular to a longitudinal direction of the carbon fibers.

2. The spinal implantable device of claim 1, wherein the spinal implantable device is a screw comprising a central shaft made of said composite material, wherein said carbon fibers stretch along a longitudinal axis of said central shaft.

3. The screw of claim 2, further comprising: threads and screw tip made of said composite material.

4. The screw of claim 2, further comprising: a coating made of a rigid material wherein said coating includes threads of said screw and screw tip of said screw.

5. The screw of claim 4, wherein the coating is made by laser welding of an outer coating layer made of said rigid material around said central shaft.

6. The screw of claim 4, wherein the coating is made by:
producing a secondary screw of the rigid material;
removing an area corresponding to said central shaft from a center of said secondary screw, leaving an outer shell made of said rigid material, wherein said outer shell comprising threads and screw tip of said screw; and
filling said outer shell with said composite material.

7. The screw of claim 4, wherein said rigid material is selectable from a list comprising: titanium, Hydroxyapatite and metal.

8. The screw of claim 4, further comprising a hole through a center of said screw, along the longitudinal axis of said screw.

9. The screw of claim 4, capable of flexing to an angle of 6 degrees.

10. The spinal implantable device of claim 1, wherein the spinal implantable device is a rod made of said composite material, wherein said carbon fibers stretch along a longitudinal axis of said rod.

11. The rod of claim 10, capable of flexing to an angle of 6 degrees.

12. The rod of claim 10, further comprising a joint.

13. The spinal implantable device of claim 1, wherein the spinal implantable device is a cup made of said composite material, wherein said carbon fibers stretch along a circumference of said cup.

14. The spinal implantable device of claim 1, wherein the spinal implantable device is a plate made of said composite material, wherein said carbon fibers stretch along a longitudinal axis of said plate.
